(19) United States
(12) Patent Application Publication
(10) Pub. No.: US 2015/0025606 A1
(43) Pub. Date: Jan. 22, 2015

(54) PILLOW WITH INTEGRATED COOLING SYSTEM

(71) Applicant: James Elliott Davis, Haslet, TX (US)

(72) Inventor: James Elliott Davis, Haslet, TX (US)

(21) Appl. No.: 14/337,102

(22) Filed: Jul. 21, 2014

Related U.S. Application Data
(60) Provisional application No. 61/856,595, filed on Jul. 19, 2013.

(51) Int. Cl. A61F 7/08

(52) U.S. Cl. CPC A61F 7/08; A61F 2007/0054
USPC 607/104

(57) ABSTRACT

The present invention relates to devices and methods that use cooling and/or heating systems in pillows. More specifically, the present disclosure relates to systems that can constantly transport a fluid throughout a pillow in order to keep the fluid at a desired temperature. In order to start the cooling/heating process, a cooling/heating device can use fluid from a tank or reservoir that can be cooled/heated by a radiator. This fluid can then exit the system, be filtered through a hose or line, and then enter the pillow itself. After flowing through the entire pillow, the fluid can be transported out of the pillow and back to the cooling/heating device. By utilizing such a design, the cooling/heating system can keep the fluid moving throughout the pillow and at a desired temperature. As such, the pillow can also maintain a corresponding desired temperature.

This application claims the benefit of U.S. Provisional Application No. 61/856,595, filed Jul. 19, 2013.

### BACKGROUND OF THE INVENTION

1. Field of the Invention

The present invention relates generally to devices and methods for pillows with cooling/heating systems and more particularly to innovative systems that use cooled/heated fluid that is filtered and transported throughout a system in order to cool/heat a pillow.

2. Description of the Related Art

Many different types of devices that assist in the process of heating and/or cooling are known in the art. Some of these devices have been applied in the form of a pad, while others have been directed for use in a pillow. One common method for heating or cooling a pillow is through the use of water. Once inside the pillow, the water is heated or cooled in an effort to heat or cool the entire pillow. However, one major problem with these types of systems is difficulty in maintaining a steady water temperature.

One method of cooling the water has been to use ice as the cooling agent. However, when exposed to room temperatures, ice will melt and eventually the temperature of the remaining water will increase. Another method for cooling a pillow has been to use coolant, but coolant also needs to be chilled before use and can quickly lose its desired temperature. Other pillow cooling devices have used air conditioning mechanisms to cool the water in the pillow, but these devices are often too large, too loud, too inconvenient, and/or too expensive. Likewise, in pillow heating devices, mechanisms to heat water throughout the pillow have been devised, but these heating devices have encountered similar problems as their pillow cooling counterparts.

Accordingly, there is a present need for a novel and efficient design for a cooling/heating system for use in pillows, which specifically deals with the aforementioned problems.

### SUMMARY OF THE INVENTION

Described herein are devices and methods that utilize cooling and/or heating systems for use in pillows. In order to start the cooling/heating process, a cooling/heating device can use fluid from a tank or reservoir that can be cooled/heated by a radiator. This fluid can then exit the cooling/heating device, filter through a hose or line, and then enter the pillow itself. After flowing through the entire pillow, the fluid can be pumped out of the pillow and back to the cooling/heating device. By utilizing such a design, the cooling/heating system can circulate the fluid throughout the pillow while the fluid temperature is maintained at a desired level.

These and other aspects and advantages of the invention will become apparent to those skilled in the art from the following detailed description and the accompanying drawings, which illustrate by way of example the features of the invention.

### BRIEF DESCRIPTION OF THE DRAWINGS

FIG. 1 is a perspective view of one embodiment of a pillow incorporating features of the present invention;

FIG. 2 is a sectional view of another embodiment of a pillow incorporating features of the present invention;

FIG. 3 is a perspective view of one embodiment of a cooling/heating device incorporating features of the present invention;

FIG. 4 is a sectional view of another embodiment of a cooling/heating device incorporating features of the present invention;

FIG. 5 is a sectional view of another embodiment of a cooling/heating device incorporating features of the present invention;

FIG. 6 is a sectional view of another embodiment of a pillow incorporating features of the present invention; and

FIG. 7 is a sectional view of another embodiment of a pillow incorporating features of the present invention.

### DETAILED DESCRIPTION OF THE INVENTION

Described herein are devices and methods that use cooling and/or heating systems in pillows. More specifically, the present disclosure relates to systems that can constantly circulate or transport a fluid throughout a pillow in order to keep that fluid and, thus, the pillow at a desired temperature. In some embodiments, to start the cooling/heating process, a cooling/heating device can use fluid from a tank or reservoir which can be cooled/heated by a radiator. After being cooled/heated, the fluid can exit the cooling/heating device and can be filtered and/or transported through a hose or line. This hose or line can be flexible, so that it can be compressed and/or twisted without stopping the flow of the fluid.

Once transported through the hose or line, the fluid can enter the pillow itself. The cooled/heated fluid can flow throughout the pillow, in order to sufficiently cool/heat the pillow. After flowing through the entire pillow, the fluid can be transported out of the pillow through the same hose or line, or an additional hose or line. This hose or line can then transport the fluid back into the cooling/heating device. By utilizing such a design, the cooling/heating system can keep the fluid constantly moving throughout the pillow. As such, the fluid can maintain a desired temperature, which can allow the pillow to likewise maintain a corresponding desired temperature.

Because the cooling/heating system utilizes constant fluid flow, the system may need a constant source of electricity. This electricity source can be an electrical outlet or a battery source, for example. In some embodiments, the present invention can serve as a complement to a conventional pillow. Yet in other embodiments, the present invention can use its own pillow, along with a cooling/heating device.

Throughout this disclosure, the preferred embodiment and examples illustrated should be considered as examples, rather than as limitations on the present invention. As used herein, the term “invention,” “device,” “apparatus,” “method,” “present invention,” “present device,” “present apparatus” or “present method” refers to any one of the embodiments of the invention described herein, and any equivalents. Furthermore, reference to various feature(s) of the “invention,” “device,” “apparatus,” “method,” “present invention,” “present device,” “present apparatus” or “present method” throughout this document does not mean that all claimed embodiments or methods must include the referenced feature(s).

It is also understood that when an element or feature is referred to as being “on” or “adjacent” to another element or feature, it can be directly on or adjacent the other element or feature or intervening elements or features may also be present. In contrast, when an element is referred to as being “directly on” or extending “directly onto” another element, there are no intervening elements present. Additionally, it is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Furthermore, relative terms such as “outer,” “above,” “lower,” “below,” “horizontal,” “vertical” and similar terms may be used herein to describe a relationship of one feature to another. It is understood that these terms are intended to encompass different orientations in addition to the orientation depicted in the figures.

Although the terms first, second, etc. may be used herein to describe various elements or components, these elements or components should not be limited by these terms. These terms are only used to distinguish one element or component from another element or component. Thus, a first element or component discussed below could be termed a second element or component without departing from the teachings of the present invention. As used herein, the term “and/or” includes any and all combinations of one or more of the associated list items.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. For example, when the present specification refers to “a” compensator, it is understood that this language encompasses a single compensator or a plurality or array of compensators. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

It is understood that the while the present disclosure makes reference to cooling/heating systems for use in pillows, and that devices which cool and/or heat pillows are the primary application concerned with the present invention, devices incorporating features of the present invention can be utilized with any application that has components or elements which aid in cooling and/or heating.

Embodiments of the invention are described herein with reference to different views and illustrations that are schematic illustrations of idealized embodiments of the invention. As such, variations from the shapes of the illustrations as a result, for example, of manufacturing techniques and/or tolerances are expected. Embodiments of the invention should not be construed as limited to the particular shapes of the regions illustrated herein but are to include deviations in shapes that result, for example, from manufacturing.

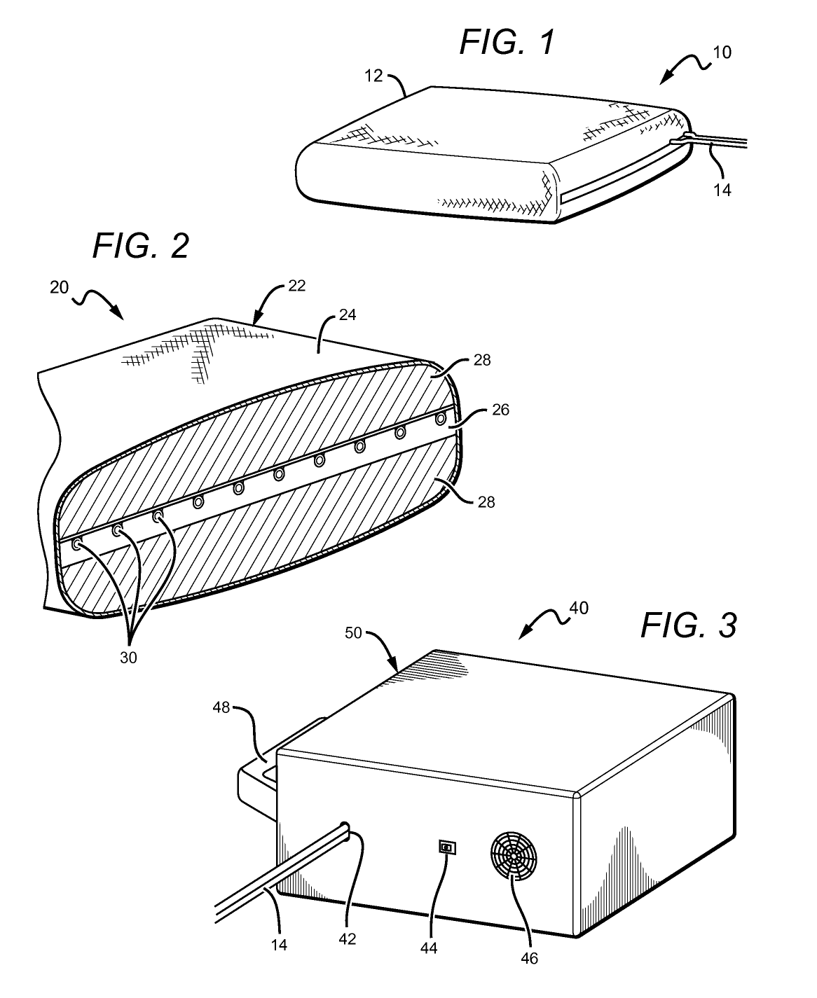

FIG. 1 is a perspective view of one embodiment of a pillow system 10 according to the present invention. The system 10 can be cooled and/or heated to a desired temperature. As described above, the present invention can effectively maintain the desired temperature by transporting a fluid throughout the system 10. In some embodiments, the system 10 can be used in cooperation with a conventional pillow. In this particular embodiment, the system 10 includes its own pillow padding 12.

FIG. 1 also displays a hose 14 that can be attached to the pillow 12. As mentioned above, the hose can take a fluid from an external cooling/heating device, and transport the fluid into the pillow 12 itself. In addition to transporting the fluid, the hose 14 can filter the fluid as well. This hose 14 can be flexible, so that it can be compressed and/or twisted without unduly restricting the flow of the fluid. Therefore, the hose 14 can comprise any material that can be compressed and/or twisted, such as Teflon®, rubber, or any material with similar characteristics.

Once the hose 14 has transported the fluid inside the pillow 12, the fluid can flow throughout a path traversing the entire pillow. By doing so, the fluid can more effectively keep the entire pillow cooled/heated to a desired temperature. In one embodiment, the fluid flows through the pillow in a snaking pattern. However, it is understood that the fluid can flow through the pillow in any manner that is efficient in keeping the fluid moving and maintaining a desired temperature.

It is also understood that the present invention can use any element or material to flow throughout the system and keep the pillow cooled/heated; any type of fluid may be used. For example, embodiments of the present invention can use water, coolant, or any other fluid, including gels, air, or other gases.

It is further understood that the basic embodiment of FIG. 1 is but one of several possible embodiments according to the present invention. FIG. 2 exhibits a sectional view of another embodiment of a pillow system 20. As depicted in FIG. 2, a pillow 22 can comprise a fabric covering 24, a center material 26, and pads 28. The fabric covering 24 can be made of a variety of fabrics or materials that serve the intended purpose of covering the pillow. In some embodiments, the center material can comprise cotton or other materials. The pad 28 can comprise foam, memory foam, or any other material that will cushion the pillow and help the pillow maintain its desired shape.

FIG. 2 also comprises internal tubes 30 that can transport the fluid throughout the pillow. These tubes 30 can be made of any material that can transport a fluid and have the fluid maintain a desired temperature. The tubes 30 should also be flexible, such that they can be compressed and/or twisted without stopping the flow of the fluid. For example, the tubes 30 can comprise a Teflon® material, plastic, rubber, or other materials.

FIG. 3 is a perspective view of one embodiment of an external cooling/heating device 40 for use in embodiments of the present invention. As shown in FIG. 3, the cooling/heating device 40 comprises a hole 42 for the hose 14 to enter the cooling/heating device 40. The cooling/heating device 40 can also comprise a tank or reservoir (not shown) that can store the fluid prior to the cooling/heating process. The cooling/heating device 40 can further comprise a radiator that can cool and/or heat the fluid. Additionally, the cooling/heating device 40 can comprise a switch 44, which allows a user to turn the device into a cooling mode or a heating mode. Furthermore, the cooling/heating device 40 can comprise a fan 46 to assist in the process of cooling/heating. Additionally, the cooling/heating device 40 can function in a manner that is similar to a home air conditioning system.

As mentioned above, because the cooling/heating device 40 utilizes constant fluid flow, the system may need a constant source of electricity. This electricity source can come from plugging the system into an electrical outlet, or from a battery source. The cooling/heating device 40 can also be portable, so it can comprise a handle 48, as well as a housing 50 that makes the device easy to transport. Furthermore, the cooling/heating device 40 can be designed to stow underneath an object, such as a bed, for easy storage out of sight. As such, the cooling/heating device 40 can have the shape and dimensions of a small box or briefcase. However, it is understood that the shape and size of the cooling/heating device are not limited to any shape and dimensions described herein, and can be any shape and size that serves the intended purpose.

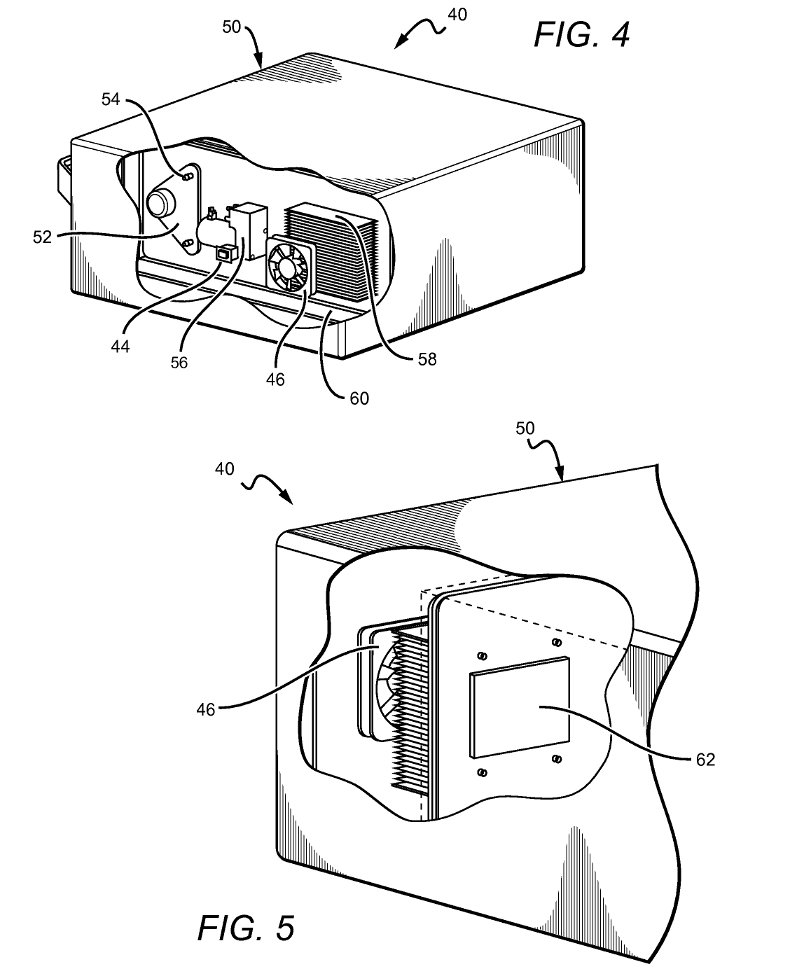

FIG. 4 exhibits a cutaway view of the cooling/heating device 40 according to the present invention. The cutaway vies exposes some of the internal elements of the device 40. FIG. 4 displays that the cooling/heating device 40 can comprise a reservoir 52, a fluid outlet 54, a fluid pump 56, a radiator 58, the fan, and insulation. The fluid outlet 54 can help transport the fluid from the hose 14 to the reservoir 52. The fluid pump 56 can pump the fluid from the reservoir 52 to the radiator 58, as well as throughout the cooling/heating system 10. It is understood that the radiator 58 and fan 46 can be used for both cooling and heating. Additionally, insulation 60 can be included to help insulate the cooling/heating device 40 so that it can maintain the fluid at a desired temperature. Although the insulation 60 can comprise foam, it can also comprise other insulative materials.

FIG. 5 shows a perspective view of a portion of the cooling/heating device 40 that may be used in embodiments of the present invention. Some of the surfaces of the device housing are made translucent to for illustrative purposes. Specifically, FIG. 5 exhibits that the cooling/heating device can also comprise a cooling/heating plate 62 to help cool/heat the fluid so that it maintains a desired temperature.

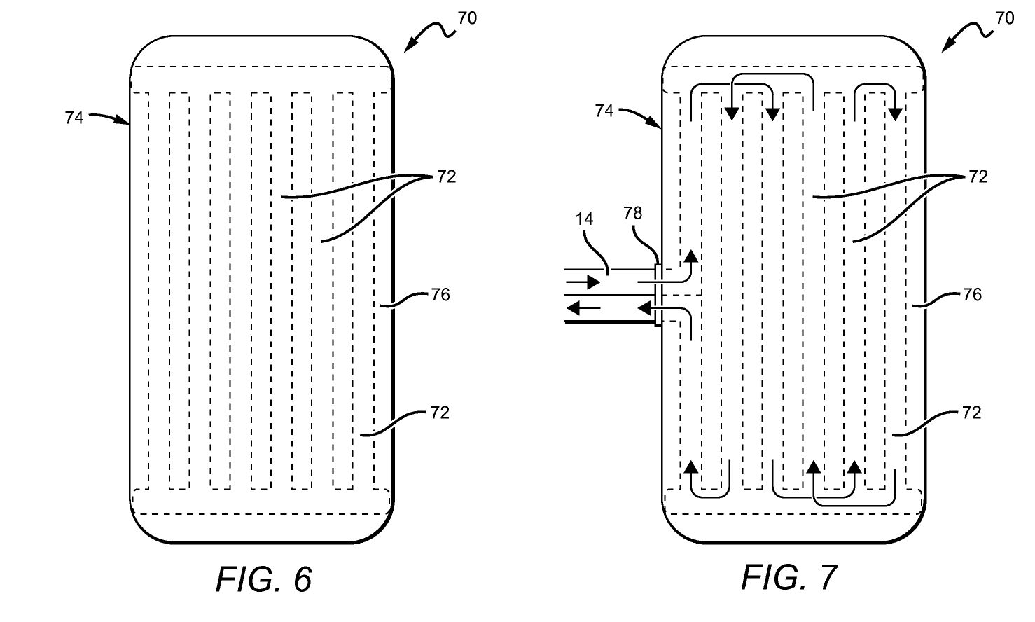

FIG. 6 displays a top plan view of another embodiment of a pillow system 70 according to embodiments of the present invention. The dashed-lines illustrate tubes 72 that transport the fluid throughout the body of the pillow 74. As mentioned above, the tubes 72 can be flexible and comprise a material such as Teflon®, plastic, or rubber. Within the pillow 74, therapeutic foam may be used to provide volume and surround the tubes 72, providing stability or the fluid transfer system.

As mentioned previously, the pillow 74 can also comprise a pillow cover. The pillow cover can be made from cotton or another material. In some embodiments, the cover material can comprise a water resistant material or layer to prevent leaks in the event that one of the interior tubes 72 ruptures. Additionally, the pillow cover can comprise a fastener, such as a zipper, for example. The pillow system 70 can also be attached to the hose 14 with a swiveling connector 78 that allows the pillow to rotate freely about the connection without binding or tangling the hose 14. Furthermore, the pillow can comprise a filler material 76, such as cotton, memory foam, or any other material which is comfortable and/or can conform to the shape of a user's head.

FIG. 7 is a sectional view of the pillow system that includes arrows indicating an exemplary fluid flow pattern of the system 70. FIG. 7 shows that the fluid can flow through the tubes 72 and throughout the pillow 74 in a back-and-forth “S” shaped pattern (i.e., a serpentine path), and then can be transported out of the pillow 74 and back to the cooling/heating device 40. In this particular embodiment, the fluid flows in a serpentine pattern, but it is understood that other patterns may also be used to circulate fluid through the pillow 74.

It is understood that embodiments presented herein are meant to be exemplary. Embodiments of the present invention can comprise any combination of compatible features shown in the various figures, and these embodiments should not be limited to those expressly illustrated and discussed. The foregoing specification and drawings are intended to cover all modifications and alternative constructions falling within the spirit and scope of the invention as expressed in the appended claims, wherein no portion of the disclosure is intended, expressly or implicitly, to be dedicated to the public domain if not set forth in the claims.

I claim:

1. A pillow system, comprising:
a pillow;
a cooling/heating device; and
at least one tube attachable between said pillow and said cooling/heating device configured to circulate a fluid between said pillow and said cooling/heating device.

2. The pillow system of claim 1, further comprising a fluid for circulation in said at least tube between said pillow and said cooling/heating device.

3. The pillow system of claim 2, said fluid comprising a coolant.

4. The pillow system of claim 1, wherein said at least one tube provides a path through said pillow for circulating a fluid there through.

5. The pillow system of claim 1, wherein said at least one tube provides a serpentine path through said pillow for circulating a fluid there through.

6. The pillow system of claim 1, further comprising a housing around said cooling/heating device.

7. The pillow system of claim 1, further comprising a filler material inside said pillow and surrounding portions of said at least one tube inside said pillow.

8. The pillow system of claim 1, wherein said pillow comprises a water resistant cover.

9. The pillow system of claim 1, said cooling/heating device further comprising:
a reservoir for holding a fluid;
a radiator for cooling or heating said fluid; and
an on/off switch.

10. The pillow system of claim 1, wherein said cooling/heating device comprises a power cord for connection to an external power source.

11. The pillow system of claim 1, wherein said cooling/heating device comprises a battery.

12. A pillow, comprising:
a cover;
a filler material inside said cover; and
at least one tube running in a pattern through said filler material inside said pillow, said at least one tube configured to circulate a fluid throughout said pillow.

13. The pillow of claim 12, wherein said at least one tube is connectable to an external cooling/heating device.

14. The pillow of claim 12, wherein said at least one tube provides a serpentine path through said pillow for circulating a fluid there through.

15. The pillow of claim 12, said cover comprising a water resistant material.

16. A method of cooling/heating a pillow, comprising:
cooling or heating a fluid;
circulating said fluid through a tube running through said pillow to adjust the temperature of material surrounding said tube within said pillow.

17. The method of claim 16, wherein said fluid is cooled or heated external to said pillow.

18. The method of claim 17, further comprising transporting said fluid to said pillow.
